(19) United States
(12) Patent Application Publication
(10) Pub. No.: US 2009/0024080 A1
(43) Pub. Date: Jan. 22, 2009

(54) ATTACHMENT FOR A MILK PUMP

(76) Inventor: Peter Rohrig, Wien (AT)

Correspondence Address: LADAS & PARRY LLP, 224 SOUTH MICHIGAN AVENUE, SUITE 1600, CHICAGO, IL, 60604, US

(21) Appl. No.: 12/161,976

(22) PCT Filed: Jan. 10, 2007
(86) PCT No.: PCT/AT07/00006

§ 371 Date: Jul. 24, 2008

(30) Foreign Application Priority Data
Jan. 24, 2006 (AT) GM 46/2006

(51) Int. Cl. A61M 1/06; A61H 1/00

(52) U.S. Cl. 604/74; 601/23

(57) ABSTRACT

An attachment for a milk pump including a position section which tapers in a funnel-shaped manner from a joining opening to a securing section which connects to the milk pump. The attachment is essentially dimensionally stable and at least one massage area is provided in positioning section, and manual massage pressure can be applied to the breast when said attachment is placed on said breast.

The invention relates to an attachment for a milk pump, including an application portion tapering in a funnel-shaped manner from an application opening to a fixing portion provided for connection with the milk pump.

These days, the pumping of breast milk by the aid of a milk pump is common and done for various reasons. On the one hand, to eliminate inconveniences for the mother when her breasts are filled with milk and sometimes even cause pain. On the other hand, a mother can take her milk at any time by the aid of a milk pump for somebody else to feed her baby with the breast milk a later time. In addition, the use of a breast pump enables a mother suffering from acute breast inflammation or sore nipples to feed breast milk nevertheless. Besides, breast milk is the best form of nutrition for prematurely born babies, increasing the chance of survival of premature babies.

During ingestion via the breast, babies take in the milk by what is called “stripping” (milking), i.e. a drawing movement by which the baby presses the breast milk out into the region of the breast nipple openings with its hard palate in the surroundings of the areola. The quality of breast milk is especially high because of the nutrients and milk fats contained therein, the fats being in fact stripped off on the inner side of the milk lines. This is only feasible by the natural stripping movement of a baby or by manual squeezing by the mother. If just vacuum is applied to a mother's breast, as is the case with most milk pumps, the milk is sucked off only with a low portion of nutrients and milk fats, and, in addition, the application of a high negative pressure may be painful to the mother. Another drawback of all milk pumps, moreover, is the fact that they are very cumbersome and awkward to handle.

From the prior art, different funnel-shaped attachments have already been known, in which elastic inserts including recesses are inserted to achieve a massage of the mother's breast by a peristaltic movement of the elastic insert. Such inserts for milk pump attachments are, for instance, shown in EP 1 107 803 B and EP 0 727 234 B. Similarly, GB 2380411 A and US 2004/0133151 A each disclose an attachment with an attachment portion in which a soft insert is received. In practice, it has however been shown that elastic inserts will only apply insufficient massage pressures to the mother's breast.

From U.S. Pat. No. 4,772,262 an electrically operated breast pump is, moreover, known, which comprises a fully elastic attachment made of a soft, deformable material. There, it is, in particular, disadvantageous that the entire attachment will deform under the vacuum produced by the pump, thus only insufficiently transmitting the pumping pressure.

From US 2005/0256449 A, a milk pump with a two-part external attachment is known, the two parts being held together via an internal elastic part. The application of a manual massage pressure is not possible there.

It is, therefore, the object of the present invention to provide an attachment by which the natural drawing movement during the ingestion of milk by a baby can be simulated as best as possible when using the attachment. In addition, the attachment is to provide greater ease of use when using the milk pump and the attachment, respectively.

In accordance with the invention, the attachment of the initially defined kind is characterized in that the attachment is designed to be substantially dimensionally stable, wherein, in the application portion, at least one massage area is provided, in which a manual massage pressure is applicable to the mother's breast when said attachment is placed on the mother's breast. The provision of at least one massage area in the application portion of the dimensionally stable attachment placed on the mother's breast, at the same time, enables the manual application of a massage pressure to the mother's breast in addition to the vacuum applied by the milk pump, so as to largely simulate the stripping movement performed by a baby with its hard palate during ingestion.

A structurally simple configuration of a massage area will be ensured if at least one perforation is provided as said massage area. This will enable the user of the attachment to directly grasp the mother's breast in the region of the perforation(s) in order to apply the desired massage pressure.

In this respect, it is also conceivable that an insert made of an elastic material is received in the attachment such that said elastic insert may be grasped through the perforations to apply the massage pressure to the mother's breast via said insert.

A structurally simple configuration of the massage area is likewise provided if the massage area is made of a softer material than the remaining attachment. This will enable the regions in which the soft material is provided to be impressed so as to apply a massage pressure to the mother's breast in order to imitate the natural stripping movement during ingestion by a baby.

In this context, it is beneficial if the massage area is made of a thermoplastic elastomer, silicone, rubber or textile, wherein it has turned out to be advantageous for the application of a manual massage pressure, if the softer area has a Shore hardness A of between 20 and 70, in particular 30 and 40.

If the remaining attachment is made of polypropylene, polycarbonate or another thermoplastic material, the attachment will have sufficient dimensional stability. In this respect, it is particularly beneficial if the remaining attachment has a Rockwell hardness R of between 80 and 120, preferably substantially 105, since this will ensure that the attachment will not deflate upon application of the vacuum via the milk pump.

With a view to simplifying production while, at the same time, ensuring the reliable connection of the softer massage areas with the remaining attachment, it is beneficial if the attachment is a two-component injection-molded part.

To provide elastic massage areas via which a manual massage pressure can be applied to the mother's breast while, at the same time, safeguarding the dimensional stability of the attachment, it is also possible to make the whole attachment of the softer material, merely comprising struts made of harder material.

In view of an efficient application of the massage pressure using, for instance, the forefinger and thumb, it is advantageous if at least two, preferably oppositely located, massage areas are provided in the application portion.

In order to readily enable the spatial orientation of the application openings, it is favorable if the longitudinal axis of the funnel-shaped application portion and the rotational axis of the fixing portion are mutually adjustable or arranged at an angle deviating from 180°. Due to the angular adjustment or arrangement of the fixing portion relative to the remaining funnel-shaped application portion of the attachment, the application opening—when slipping the attachment according to the invention onto a conventional, linear suction socket—will not project out of a top portion of the milk pump in the bulky, unhandy position located substantially perpendicular to the longitudinal extension of the pump, but will enable a user-friendly adaptation of the orientation of the application opening, and hence greater ease of use, as a function of the position in which the mother uses the milk pump (e.g., seated, lying, etc.) and also as a function of from which breast milk is just being taken and whether the mother is left-hander or right-hander. Consequently, the use of an attachment according to the invention is particularly suitable in manual milk pumps; yet, it may, of course, also be employed in electrically operated milk pumps.

In order to enable the attachment to be rotated in its position inserted in a suction socket of the milk pump, and hence achieve a different orientation of the application opening, it is advantageous if the fixing portion has the shape of a rotationally symmetric rotational body. For the friction-fit engagement between the suction socket of the milk pump and the attachment, it is favorable if the fixing portion is conically designed. Alternatively, it is also possible to form the fixing portion cylindrically, in which latter case the suction socket is advantageously conically designed.

The adjustability of the spatial orientation between the rotational axis of the fixing portion and the longitudinal axis of the funnel-shaped application portion will also be readily achieved if the application portion and the fixing portion are interconnected by an articulated connection.

An articulated connection advantageously allowing for a pivotal movement by 360° will be provided if the application portion and the fixing portion are interconnected by a universal-joint connection.

In a structurally simple manner, a pivotal movement of the application portion relative to the fixing portion may likewise be achieved if the application portion and the fixing portion are interconnected by a flexible connecting portion. In this case, it is beneficial for the simple pivoting of the application portion relative to the fixing portion, if the flexible connecting portion is made of the same synthetic material as the massage area.

A high user-friendliness will, in particular, be achieved, if the longitudinal axis and the rotational axis are adjustable or arranged at an angle of between 150° and 170°, preferably substantially 165°.

In the following, the invention will be explained in even more detail by way of preferred exemplary embodiments illustrated in the drawings, to which it is, however, not to be restricted. In detail, in the drawing:

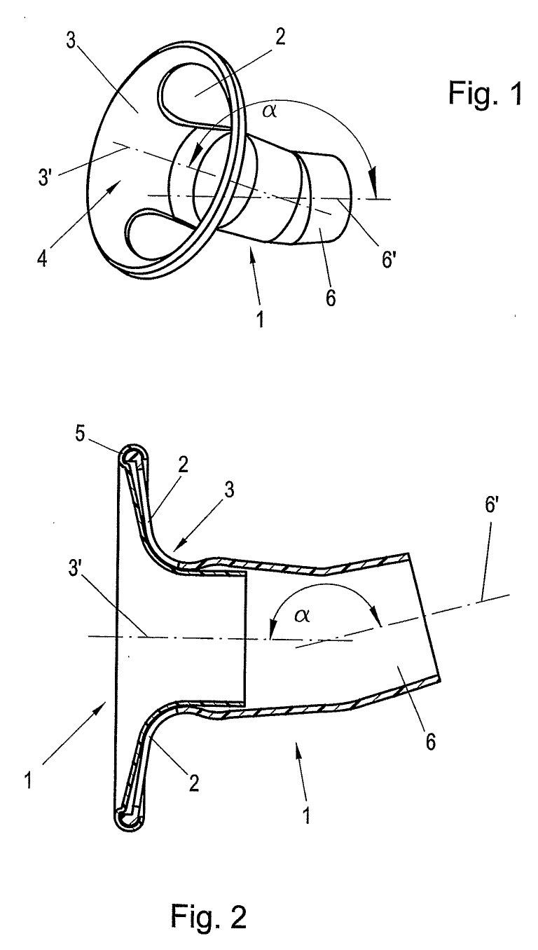

FIG. 1 is a perspective view of an attachment having two oppositely located perforations;

FIG. 2 is a section through the attachment according to FIG. 1 including an elastic insert;

FIG. 3 is a view of the attachment according to FIG. 1;

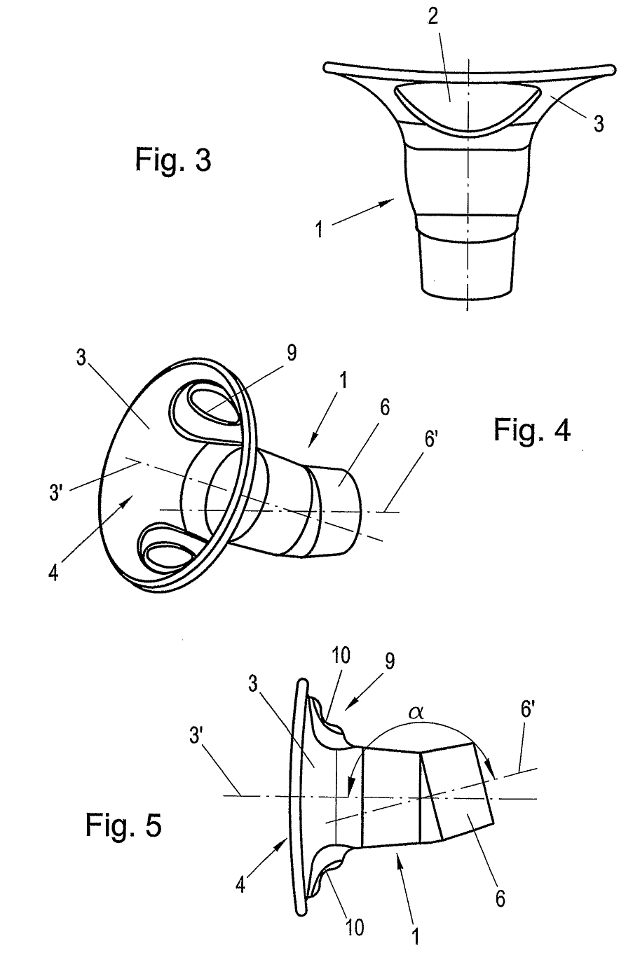

FIG. 4 is a perspective view of an attachment featuring two opposite regions of softer material;

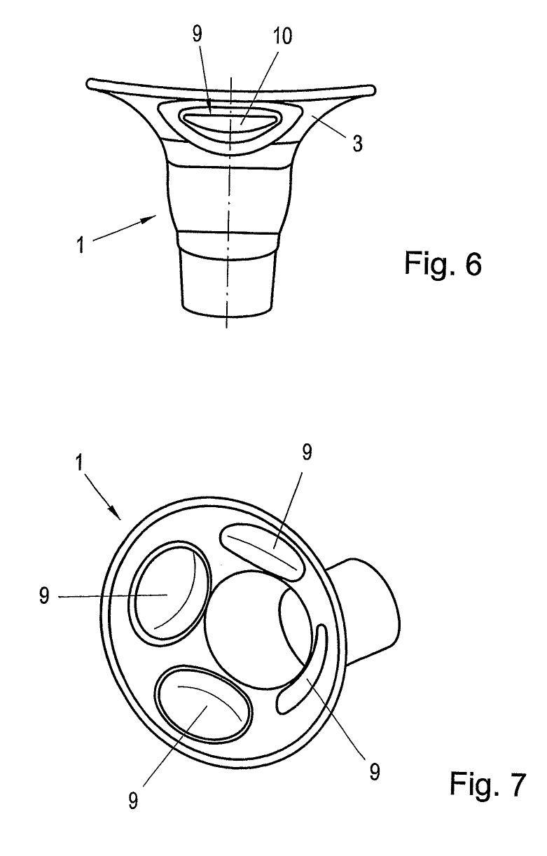

FIG. 5 is a view of the attachment according to FIG. 4;

FIG. 6 is a further view of the attachment according to FIG. 4;

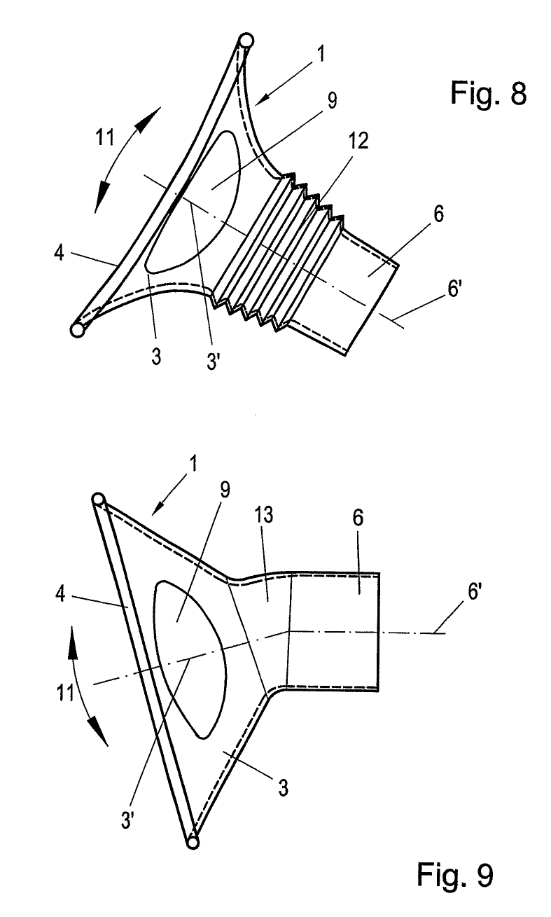

FIG. 7 is a perspective view of an attachment featuring four massage areas;

FIG. 8 is a view of an attachment in which the application portion and the fixing portion are interconnected by an articulated connecting portion folded like bellows;

FIG. 9 is a view of an attachment in which the application portion and the fixing portion are interconnected by a connecting portion made of soft plastic;

FIG. 10 is a view of an attachment in which the application portion and the fixing portion are interconnected via a universal joint;

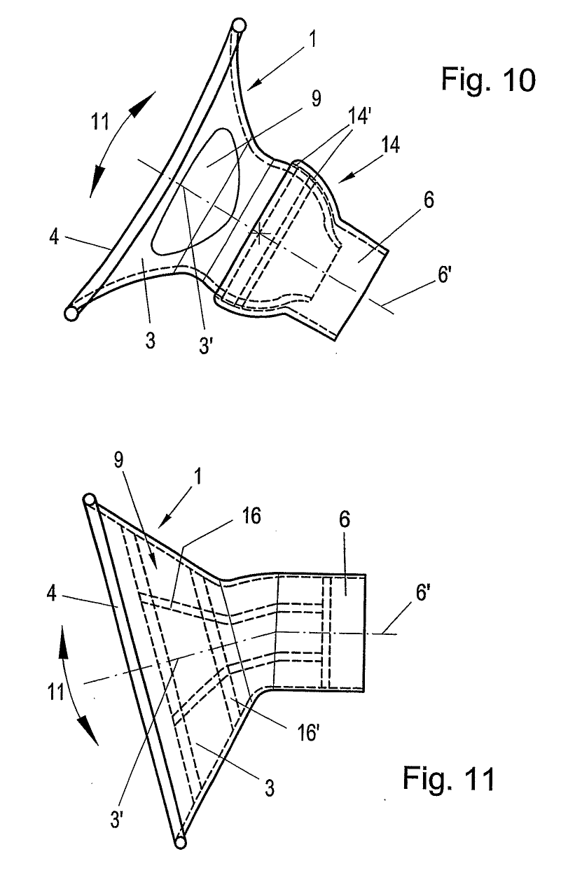

FIG. 11 is a view of an attachment made of soft plastic and reinforced by struts of harder plastic;

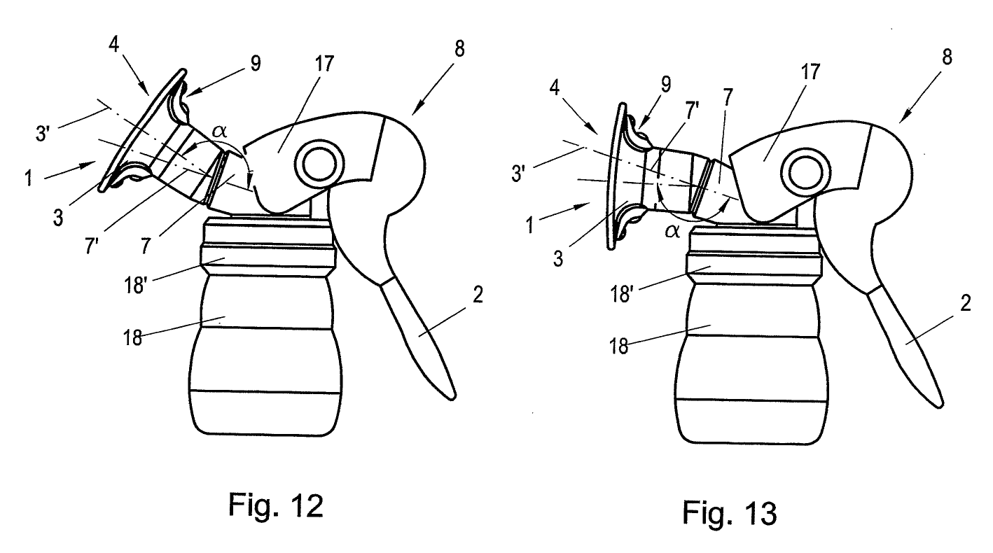

FIG. 12 is a view of a milk pump in whose suction the attachment according to FIGS. 4 to 6 is inserted; and

FIG. 13 is a view of the milk pump according to FIG. 12 with the attachment arranged in a manner rotated by 180°.

FIGS. 1 to 3 depict an attachment 1 having two oppositely located perforations 2 in a funnel-shaped application portion 3 of the attachment 1. The perforations 2, which are provided as massage areas, in a position of the attachment 1 placed on the mother's breast via an application opening 4, allow for the manual application of a massage pressure to the mother's breast, thus largely simulating the natural stripping movement of a baby.

From the sectional illustration according to FIG. 2, it is, moreover, apparent that an elastic insert 5 can be inserted in the application portion 3 of the attachment 1 to avoid direct access to the mother's breast, but provide access to the elastic insert part 5 through the perforations 2 so as to enable the massage pressure to be applied to the mother's breast via the former.

As is, in particular, apparent from FIGS. 1 and 2, a longitudinal axis 3′ of the funnel-shaped application portion 3 is arranged at an angle α relative to a rotational axis 6′ of a fixing portion 6 adjoining the application portion 3. Thus, a change in the spatial orientation of the application opening 4 by simply rotating the attachment 1 in a suction socket 7 of a milk pump 8 (cf. FIGS. 11 and 12) can be achieved. The rotational axis 6′ of the fixing portion 6 may, of course, also be arranged coaxially with the longitudinal axis 3′ of the application portion 3.

FIGS. 4 to 6 illustrate an alternative embodiment, in which two oppositely located massage areas are likewise provided, wherein, however, no perforations 2 but areas 9 are provided, in which the suction socket 7 is made of a softer material than the remaining suction socket. In these massage areas 9 of softer material, which may, for instance, comprise a thermoplastic elastomer, silicone, rubber or textile, it is, thus, feasible in a simple manner to apply a massage pressure to the mother's breast by an elastic deformation of the soft material. As is particularly apparent from FIG. 5, the outer surfaces of the soft massage areas 9 may each comprise an indentation 10 such that a mother using the attachment 1 does not slip off the soft massage areas 9 during the application of the massage pressure, but, for instance, her finger and thumb are guided in the indentations 10 for the purpose of applying the massage pressure.

FIG. 7 depicts a further exemplary embodiment, in which massage areas 9 each arranged in a manner offset by 90° are provided.

In FIGS. 8 to 10, three exemplary embodiments of the attachment 1 are illustrated, in which the application portion 3 is arranged to be pivotable relative to the fixing portion 6, i.e. the angle between the longitudinal axis 3′ of the attachment 3 and the rotational axis 6′ of the fixing portion 6 is changeable by pivoting in the sense of arrow 11. In the application portion 3, areas 9 of softer material are each visible as massage areas via which a massage pressure can be applied to simulate the natural stripping movement of an infant.

In the exemplary embodiment represented in FIG. 8, a connecting portion 12 folded like bellows is provided between the fastening portion 6 and the application portion 3 to achieve pivotability.

By contrast, FIG. 9 illustrates a connecting portion 13 which, like the soft massage areas 9, is made of a softer plastic than the remaining attachment part 1 so as to enable a pivotal movement of the application portion 3 relative to the fixing portion 6 in the sense of arrow 11 on account of the elasticity of the material in the connecting portion 13.

By contrast, in FIG. 10, the pivotability of the application portion 3 relative to the fixing portion 6 is achieved by the aid of a universal-joint-like connection 14, with two sealing rings 14′ being arranged between the application portion 3 and the fixing portion 6.

FIG. 11 depicts a further exemplary embodiment of an attachment 1 for a milk pump 8, in which the entire attachment 1 is substantially comprised of an elastic material such that a massage pressure can be applied to the mother's beast in these areas 9. In order to achieve a dimensional stability of the attachment 1, and hence prevent its deflation due to the negative pressure applied by the milk pump, the attachment 1 comprises struts 16 extending in its longitudinal direction and radially extending struts 16′ of harder plastic than the attachment funnel is usually made, such as, e.g., polycarbonate, polypropylene or even other thermoplastic materials having a Rockwell hardness of between 80 and 120 R.

In FIGS. 12 and 13, the attachment is illustrated in its position inserted in a suction socket 7 of the milk pump 8. In this case, the milk pump 8 comprises a manual pumping device 17 as well as a milk container 18 to which the milk pump 8 is fastened via a screw cap 18′. It is, in particular, apparent that, due to the angular orientation of the longitudinal axis 3′ of the application portion 3 relative to a longitudinal axis 7′ of the suction socket 7, the orientation of the application opening 4 and also of the softer massage areas 9 can be readily changed by simply rotating the attachment 1 within the suction socket 7. Thus, an especially high ease of use is provided when using the milk pump and its attachment.

What is, however, essential is that massage areas 2, 9 are provided, in which a massage pressure can be applied to the mother's breast when the attachment 1 is attached to the breast, in order to imitate the natural drawing movement (“stripping”) during the ingestion of milk by an infant when using the milk pump.

1. An attachment for a milk pump, including an application portion tapering in a funnel-shaped manner from an application opening to a fixing portion provided for connection with the milk pump, wherein the attachment is designed to be substantially dimensionally stable, wherein, in the application portion, at least one massage area is provided, in which a manual massage pressure is applicable to the mother's breast when said attachment is placed on the mother's breast.

2. An attachment according to claim 1, wherein at least one perforation is provided as said massage area.

3. An attachment according to claim 2, wherein an insert made of an elastic material is received in the attachment.

4. An attachment according to claim 1, wherein the massage area is made of a softer material than the remaining attachment.

5. An attachment according to claim 4, wherein the massage area is made of a thermoplastic elastomer, silicone, rubber or textile.

6. An attachment according to claim 4, wherein the softer area has a Shore hardness A of between 20 and 70.

7. An attachment according to claim 4, wherein the remaining attachment is made of polypropylene, polycarbonate or another thermoplastic material.

8. An attachment according to claim 4, wherein the remaining attachment has a Rockwell hardness R of between 80 and 120.

9. An attachment according to claim 4, wherein the attachment is a two-component injection-molded article.

10. An attachment according to claim 4, wherein the entire attachment is made of the softer material, merely comprising struts made of harder material.

11. An attachment according to any one of claims 1 to 10, claim 1, wherein at least two massage areas are provided in the application portion.

12. An attachment according to claim 1, wherein a longitudinal axis of the funnel-shaped application portion and a rotational axis of the fixing portion are mutually adjustable or arranged at an angle deviating from 180°.

13. An attachment according to claim 1, wherein the fixing portion has the shape of a rotationally symmetric rotational body.

14. An attachment according to claim 13, wherein the fixing portion is conically designed.

15. An attachment according to claim 13, wherein the fixing portion is cylindrically designed.

16. An attachment according to claim 15, wherein the application portion and the fixing portion are interconnected by an articulated connection.

17. An attachment according to claim 16, wherein the application portion and the fixing portion are interconnected by a universal-joint connection.

18. An attachment according to claim 16, wherein the application portion and the fixing portion are interconnected by a flexible connecting portion.

19. An attachment according to claim 18, wherein the flexible connecting portion is made of the same synthetic material as the massage area

20. An attachment according to claim 15, wherein the longitudinal axis and the rotational axis are arranged or adjustable at an angle of between 150° and 170°.
